(19) United States
(12) Patent Application Publication
(10) Pub. No.: US 2026/0062363 A1
(43) Pub. Date: Mar. 5, 2026

(54) PROCESS OF CONTROLLING PROVISION OF A HYDROGEN STREAM

(71) Applicant: UOP LLC, Rosemont, IL (US)

(72) Inventors: Ian G. Horn, Streamwood, IL (US); James W. Harris, Palatine, IL (US); Srinivasan Ramanujam, Bengaluru (IN)

(21) Appl. No.: 19/270,068

(22) Filed: Jul. 15, 2025

Related U.S. Application Data
(60) Provisional application No. 63/688,092, filed on Aug. 28, 2024.

(51) Int. Cl. C07C 5/32; C01B 3/00

(52) U.S. Cl. CPC C07C 5/32; C01B 3/0015

(57) ABSTRACT

A process of providing a hydrogen stream is disclosed. The process comprises charging a hydrocarbon feed stream to a dehydrogenation reactor to provide a dehydrogenated stream. The dehydrogenated stream is separated in a separator to provide a vapor stream comprising hydrogen and a liquid stream. A hydrogen product stream is taken from the vapor stream. The hydrogen product stream is fed to a hydrogen feed line. An outlet pressure of the hydrogen product stream in the hydrogen feed line is compared with a set point pressure. Based on a comparison of the outlet pressure with the set point pressure, a flow rate of the hydrocarbon feed stream to the dehydrogenation reactor is modulated. The present process responds to the demand of hydrogen by adjusting the production of hydrogen in the process and its supply from the hydrogen feed line.

### FIELD

The field is the process of providing a hydrogen stream. The field may particularly relate to the process of providing a hydrogen stream based on hydrogen demand.

### BACKGROUND

Hydrogen is expected to have significant growth potential because it is a clean-burning fuel. However, hydrogen production processes based on steam reforming, autothermal reforming, partial oxidation, or gasification of hydrocarbon feedstocks are significant emitters of carbon dioxide. Government regulations and societal pressures are increasingly taxing or penalizing carbon dioxide emissions or incentivizing carbon dioxide capture. Consequently, there is significant interest in lowering the cost of hydrogen production and recovering the byproduct carbon dioxide. The renewed interest in alternative energy sources and energy carriers opens up new prospects for providing hydrogen feed for fuel cells, power generation and many more applications.

Increased global demand for hydrogen requires new modes for transporting hydrogen especially to locations which are hydrogen depleted. Hydrogen generated by renewable energy sources is called green hydrogen. Hydrogen is expected to be an important element in the future fuel economy and may need to be transported to locations as far as 8000 km from the source of generation to remote hydrogen depleted regions.

There exists a huge regional disparity in the cost for production of hydrogen. A number of technologies have been developed for transporting hydrogen, including ammonia, liquid hydrogen, and liquid organic hydrogen carrier (LOHC) to address this disparity. Toluene-methylcyclohexane (MCH) is expected to be a significant player in LOHC processes considering its easy integration into the existing fuel sector supply chain and distribution network, utilization in idle refinery assets, flexibility for co-processing, and higher relative safety handling.

LOHC involves the reversible dehydrogenation reaction of a hydrogen carrier such as methylcyclohexane (MCH) to produce toluene and hydrogen. It has been proposed as a solution for the storage, transportation, and distribution of hydrogen produced from renewable or non-renewable energy sources. For power generation, the hydrogen from this process is usually compressed for a downstream power generation unit. Usually, purity requirements for a power generation unit are very stringent. Due to the relatively high cost associated with green hydrogen production, it is necessary to recover almost all of the hydrogen for the process to be economical.

Hydrogen produced in the LOHC process may be exported to other processes or units that require hydrogen such as a refinery. The requirement of hydrogen in the refinery may vary according to the demand of hydrogen in the refinery. In the absence of a control scheme for adjusting the supply of hydrogen from the LOHC to the downstream hydrogen-consuming unit, the excess hydrogen will have to be burned or in the event of non-supply, additional hydrogen must be procured from perhaps non-green sources. So, there is a need to manage the supply of hydrogen from the LOHC to a location for use according to the demand of hydrogen in the downstream location of use, for example, to a refinery header according to the demand for hydrogen in the downstream refinery.

Accordingly, it would be desirable to have a more effective and efficient way to control hydrogen supply from an LOHC unit to a downstream hydrogen consuming plant.

### BRIEF SUMMARY

A process of providing a hydrogen stream is disclosed. The process comprises charging a hydrocarbon feed stream to a dehydrogenation reactor to provide a dehydrogenated stream. The dehydrogenated stream is separated in a separator to provide a vapor stream comprising hydrogen and a liquid stream. A hydrogen product stream is taken from the vapor stream. The hydrogen product stream is fed to a hydrogen feed line. An outlet pressure of the hydrogen product stream in the hydrogen feed line is compared with a set point pressure. Based on a comparison of the outlet pressure with the set point pressure, a flow rate of the hydrocarbon feed stream to the dehydrogenation reactor is adjusted. The present process responds automatically to changes in hydrogen demand and adjusts the hydrogen production and capacity of the dehydrogenation unit with little or no intervention from the operator.

### Definitions

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take a main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant” or “predominate” or “predominantly” means greater than 50%, suitably greater than 75% and preferably greater than 90%.

The term “Cx” is to be understood to refer to molecules having the number of carbon atoms represented by the subscript “x”. Similarly, the term “Cx-” refers to molecules that contain less than or equal to x and preferably x and less carbon atoms. The term “Cx+” refers to molecules with more than or equal to x and preferably x and more carbon atoms.

As used herein, the term “carbon number” refers to the number of carbon atoms per hydrocarbon molecule and typically a paraffin molecule.

As used herein, the term “controller” refers to an algorithm that takes a measurement and compares it to a set point, calculating an output according to one of a number algorithms, which could include a proportional calculation, a time-based integral calculation, a time-based derivative calculation, or some combination of these three.

As used herein, the term “low signal selector” refers to an algorithm that takes two or more signals and selects the smallest of them for the output.

As used herein, the term “high signal selector” refers an algorithm that takes two or more signals and selects the largest of them for the output.

As used herein, the term “duty” with respect to a heater in a heating step refers to the amount of heat the heater needs to provide to achieve a specific temperature change.

### BRIEF DESCRIPTION OF THE DRAWING

FIG. 1 is an illustration of an exemplary embodiment of a process of providing a hydrogen stream.

FIG. 2 is an illustration of another exemplary embodiment of a process of providing a hydrogen stream.

### DETAILED DESCRIPTION

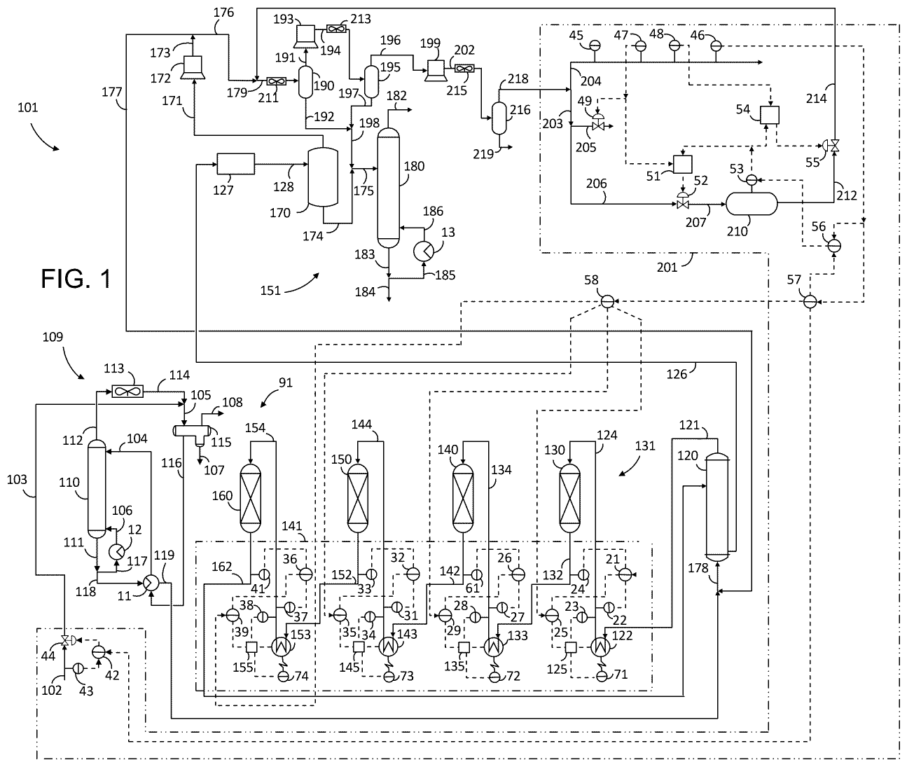

FIG. 1 illustrates an embodiment of a process 101 for providing a hydrogen stream. In an aspect, the process 101 represents the dehydrogenation cycle of producing hydrogen from a hydrogen carrier such as a LOHC. The process 101 comprises a dehydrogenation section 109, and a purification section 151. The dehydrogenation section 109 may comprise a feed preparation section 91 and a dehydrogenation reactor section 131. The dehydrogenation reactor section 131 may comprise one or more dehydrogenation reactors.

In accordance with the present disclosure, the process of providing a hydrogen stream 101 as shown in FIG. 1, further comprises a hydrogen header pressure controller 201 to control the supply of the hydrogen stream produced in the process 101 supplied to another process and/or unit. As described later in detail, the hydrogen header pressure controller 201 may comprise one or more sensors, control, valves, and switches for controlling the hydrogen flow and supply with and from the process 101. As shown, a hydrocarbon feed stream comprising a hydrogenated hydrocarbon such as methylcyclohexane may be taken in a feed line 102 and charged to the dehydrogenation reactor section 131. In an aspect, the hydrocarbon feed stream comprising a hydrogenated hydrocarbon such as methylcyclohexane in line 102 may be taken from a hydrogenation unit . . . .

The hydrocarbon feed stream in the hydrocarbon feed line 102 may be taken from an external source such as a storage tank or a tanker (both not shown). If the hydrocarbon feed stream is imported from an external source, such as through a pipeline, land-going vehicle, or water-going vehicle, the feed stream may be exposed to oxygen and require treatment to remove the oxygen and/or oxygenated hydrocarbons, prior to introduction into the dehydrogenation reactor section 131. Such oxygen removal treatments may include but are not limited to oxygen stripping, heat soaking, caustic treatment, adsorption using activated alumina and/or molecular sieves, resins, fractionation, clay treatment, or any combination thereof.

In an exemplary embodiment, the hydrocarbon feed stream in line 102 may be passed to an oxygen stripping column 110 to remove oxygen, oxygenated hydrocarbons and water from the feed stream.

As described later in detail, the hydrocarbon feed stream in line 102 may be passed through feed control valve 44 to provide a controlled flow of the feed stream which is taken in line 103. The hydrocarbon feed stream in line 103 is passed to the oxygen stripping column 110.

The hydrocarbon feed stream in line 102 may be passed to an overhead receiver 115 of the oxygen stripping column 110. In an embodiment, the hydrocarbon feed stream in line 103 may be combined with a cooled overhead stream of the oxygen stripping column in line 114 to provide a combined overhead stream in line 105. The combined overhead stream in line 105 is passed to the overhead receiver 115. From the overhead receiver 115, an overhead liquid stream is taken in line 116 and passed to the oxygen stripping column 110. Water may be separated in line 107 from the combined overhead stream and taken from a boot of the overhead receiver 115. In an embodiment, the overhead liquid stream in line 116 may be heated in a heat exchanger 11 by heat exchange with a stripped hydrocarbon feed stream in line 118 to provide a heated overhead liquid stream in line 104. The heated overhead liquid stream in line 104 is passed to the oxygen stripping column 110. In the oxygen stripping column 110, the heated overhead liquid stream in line 104 comprising the hydrocarbon feed stream in line 102 is stripped of oxygen and oxygenates. An overhead stream containing oxygen and/or oxygenates is taken in line 112, cooled in a cooler such as an air cooler 113 to provide the cooled overhead stream in line 114. In the receiver 115, oxygen is separated from the cooled overhead stream in line 114 and taken in an off-gas stream in line 108.

A stripping column bottoms stream is taken from the bottom of the oxygen stripping column 110 in line 111. A reboiling stream is taken from the stripping column bottoms stream in line 117 and heated in a reboiler 12 with steam. A reboiled stream is passed back to the oxygen stripping column 110 near the bottom in line 106. The remainder of the stripping column bottoms stream is taken as a stripped hydrocarbon feed stream in line 118. The stripped hydrocarbon feed stream in line 118 is heat exchanged with the hydrocarbon feed stream in line 103 in the heat exchanger 11 to provide a cooled stripped hydrocarbon feed stream in line 119 which is passed to the dehydrogenation reactor section 131.

In an exemplary embodiment, the dehydrogenation reactor section 131 comprises four dehydrogenation reactors, a first dehydrogenation reactor 130, a second dehydrogenation reactor 140, a third dehydrogenation reactor 150, and a fourth dehydrogenation reactor 160. Fewer or more dehydrogenation reactors than four may be utilized.

The cooled stripped hydrocarbon feed stream in line 119 may have sufficient heat for feeding into the dehydrogenation reactor section 131. In an aspect, the cooled stripped hydrocarbon feed stream in line 119 may be heat exchanged in a combined feed exchanger 120 with a fourth dehydrogenated effluent stream in line 162 to heat up the cooled stripped hydrocarbon feed stream in line 119 before passing it to the dehydrogenation reactor section 131. In an embodiment, the cooled stripped hydrocarbon feed stream in line 119 may be combined with a recycled vapor stream in line 177 to provide a combined hydrocarbon feed stream in line 178. The combined hydrocarbon feed stream in line 178 is heat exchanged in the combined feed exchanger 120 with the fourth dehydrogenated effluent stream in line 162 to produce a heated combined hydrocarbon feed stream in line 121. The heated combined hydrocarbon feed stream in line 121 is charged to the dehydrogenation reactor section 131. In an embodiment, the cooled stripped hydrocarbon feed stream in line 119 and the recycled vapor stream in line 177 may be fed to the combined feed exchanger 120 separately.

In an exemplary embodiment, the heated combined hydrocarbon feed stream in line 121 is further heated in a first feed heater 122 to provide a twice heated combined hydrocarbon feed stream in line 124. The twice heated combined hydrocarbon feed stream in line 124 is charged to the first dehydrogenation reactor 130. The first dehydrogenation reactor 130 may be operated at a temperature of about 420° C. (788° F.) to about 600° C. (1112° F.), preferably about 480° C. (896° F.) to about 600° C. (1112° F.), more preferably around 480° C. (896° F.) to about 560° C. (1040° F.) and pressure of about 620 kPa (g) (90 psig) to 758 kPa (g) (110 psig).

The first dehydrogenation reactor 130 may comprise one or more dehydrogenation catalyst bed(s). Any suitable dehydrogenation catalyst that can achieve a selectivity in the dehydrogenation of methylcyclohexane to toluene and hydrogen in excess of 99.8% can be used in the first dehydrogenation reactor 130. Suitable dehydrogenation catalysts may include, but are not limited to, alumina, a noble metal, and an alkali or alkaline earth metal. Suitable noble metals include, but are not limited to, platinum, palladium, rhodium, ruthenium, rhenium, iridium, gold, osmium, silver, or combinations thereof. Suitable alkali or alkaline earth metals include, but are not limited to, sodium, cesium, potassium, rubidium, francium, lithium, beryllium, strontium, barium, calcium, magnesium, radium, or combinations thereof.

A first dehydrogenated effluent stream comprising the dehydrogenated hydrocarbon such as toluene is taken in line 132 from the first dehydrogenation reactor 130. The first dehydrogenated effluent stream in line 132 may be further dehydrogenated in the second dehydrogenation reactor 140. The first dehydrogenated effluent stream in line 132 may be heated in a second feed heater 133 to provide a heated first dehydrogenated effluent stream in line 134. The heated first dehydrogenated effluent stream in line 134 is passed to the second dehydrogenation reactor 140. The second dehydrogenation reactor 140 may comprise one or more dehydrogenation catalyst bed(s). The second dehydrogenation reactor 140 may comprise similar or different dehydrogenation catalyst than the first dehydrogenation reactor 130. The second dehydrogenation reactor 140 may be operated at a temperature of about 420° C. (788° F.) to about 600° C. (1112° F.), preferably about 480° C. (896° F.) to about 600° C. (1112° F.), more preferably around 480° C. (896° F.) to about 560° C. (1040° F.) and pressure of about 620 kPa (g) (90 psig) to 758 kPa (g) (110 psig).

A second dehydrogenated effluent stream comprising the dehydrogenated hydrocarbon such as toluene is taken in line 142 from the second dehydrogenation reactor 140. The second dehydrogenated effluent stream in line 142 may be further dehydrogenated in the third dehydrogenation reactor 150. The second dehydrogenated effluent stream in line 142 may be heated in a third feed heater 143 to provide a heated second dehydrogenated effluent stream in line 144. The heated second dehydrogenated effluent stream in line 144 is passed to the third dehydrogenation reactor 150. The third dehydrogenation reactor 150 may comprise one or more dehydrogenation catalyst bed(s). The third dehydrogenation reactor 150 may comprise similar or different dehydrogenation catalyst than the first dehydrogenation reactor 130 and/or the second dehydrogenation reactor 140. The third dehydrogenation reactor 150 may be operated at a temperature of about 420° C. (788° F.) to about 600° C. (1112° F.), preferably about 480° C. (896° F.) to about 600° C. (1112° F.), more preferably around 480° C. (896° F.) to about 560° C. (1040° F.) and pressure of about 620 kPa (gauge) (90 psig) to 758 kPa (gauge) (110 psig).

A third dehydrogenated effluent stream comprising the dehydrogenated hydrocarbon such as toluene is taken in line 152 from the third dehydrogenation reactor 150. The third dehydrogenated effluent stream in line 152 may be further dehydrogenated in the fourth dehydrogenation reactor 160. The third dehydrogenated effluent stream in line 152 may be heated in a fourth feed heater 153 to provide a heated third dehydrogenated effluent stream in line 154. The heated third dehydrogenated effluent stream in line 154 is passed to the fourth dehydrogenation reactor 160. The fourth dehydrogenation reactor 160 may comprise one or more dehydrogenation catalyst bed(s). The fourth dehydrogenation reactor 160 may comprise similar or different dehydrogenation catalyst than the first dehydrogenation reactor 130 and/or the second dehydrogenation reactor 140 and/or the third dehydrogenation reactor 150. The fourth dehydrogenation reactor 160 may be operated at a temperature of about 420° C. (788° F.) to about 600° C. (1112° F.), preferably about 480° C. (896° F.) to about 600° C. (1112° F.), more preferably around 480° C. (896° F.) to about 560° C. (1040° F.) and pressure of about 620 kPa (gauge) (90 psig) to 758 kPa (gauge) (110 psig).

The fourth dehydrogenated effluent stream comprising the dehydrogenated hydrocarbon such as toluene and hydrogen is taken in line 162 from the fourth dehydrogenation reactor 160 and passed to the combined feed exchanger 120 to be heat exchanged with the combined hydrocarbon feed stream in line 178. A heat exchanged perhaps cooled fourth dehydrogenated effluent stream is taken in line 126 from the combined feed exchanger 126. The cooled fourth dehydrogenated effluent stream in line 126 is further cooled in a heat exchanger 127 to provide a twice cooled fourth dehydrogenated effluent stream in line 128. In an exemplary embodiment, the heat exchanger 127 may be a shell and tube heat exchanger. The reactions taking place in the dehydrogenation reactors 130, 140, 150, and 160 may include a main reaction of dehydrogenation of the hydrogenated hydrocarbon such as methylcyclohexane to toluene releasing hydrogen and may include one or more side reactions. One side reaction includes cracking of toluene to benzene and methane. Another side reaction includes isomerization of the hydrogenated hydrocarbon such as methylcyclohexane to dimethylcyclopentane (DMCP), and ethylcyclopentane (ECP). A third side reaction includes the dimerization of the hydrogenated hydrocarbon to heavier molecules. These side reactions may affect the overall yield and the purity of the products streams of the process 101. The twice cooled fourth dehydrogenated effluent stream in line 128 may be passed to the purification section 151 to purify the one or more product streams. The purification section 151 separates byproducts from the products streams to promote the purity of the product streams while consuming a minimum of energy in the separation.

The twice cooled fourth dehydrogenated effluent stream in line 128 is passed to a separator 170 to separate the dehydrogenated effluent stream comprising the dehydrogenated hydrocarbon such as toluene into vapor and liquid streams. The twice cooled fourth dehydrogenated effluent stream in line 128 is passed to the separator 170 where it is separated into a separator vapor stream comprising hydrogen in line 171 and a separator liquid stream comprising toluene in line 174. The separator vapor stream in line 171 may comprise methane and traces of C6-hydrocarbons along with the hydrogen. The separator vapor stream in line 171 may be passed to a recycle gas compressor 172 to provide a compressed separator vapor stream in line 173. In an embodiment, the recycle gas compressor 172 may compress the separator vapor stream in line 171 to pressure of about 758 kPa (g) (110 psig) to about 1379 kPa (g) (200 psig). The compressed separator vapor stream in line 173 may be split to provide a first net vapor stream in line 176 and a recycled vapor stream in line 177. In an aspect, the recycled vapor stream in line 177 is recycled to the dehydrogenation reactor section 131. In an embodiment, the recycled vapor stream in line 177 may be combined with the cooled stripped hydrocarbon feed stream in line 119 to provide the combined hydrocarbon feed stream in line 178 and passed through the combined feed exchanger 120.

The separator liquid stream in line 174 is a toluene rich liquid stream, and toluene is separated from this stream. The separator liquid stream in line 174 is further purified in the purification section 151. In an embodiment, the purification section 151 further comprises a stabilizer column 180. In an embodiment, the separator liquid stream in line 174 is combined with a combined liquid stream in line 198 to provide a combined liquid dehydrogenated stream in line 175. The combined liquid dehydrogenated stream in line 175 is passed to the stabilizer column 180. In an embodiment, the stabilizer column 180 may be operated at an overhead pressure of about 34 kPa (g) (5 psig) to about 207 kPa (g) (30 psig) and a bottoms temperature of about 65° C. (150° F.) to about 122° C. (250° F.). In an aspect, the heat to the stabilizer column 180 may be supplied from a reboiler 13 using steam. An overhead stream comprising dehydrogenated hydrocarbon is taken in line 182 from the stabilizer column 180. A bottoms stream rich in dehydrogenated hydrocarbon is taken in a bottoms line 183 from the stabilizer column 180. A reboiling stream may be taken in line 185 from the bottoms stream. The reboiling stream in line 185 is heated in the reboiler 13 with steam to provide a reboiled stream in line 186. The reboiled stream in line 186 is recycled to the stabilizer column 180 near the bottom of the column. A dehydrogenated hydrocarbon stream is taken in line 184 from the bottoms of the stabilizer column 180. In an exemplary embodiment of the present disclosure, the dehydrogenated hydrocarbon is toluene. In an aspect, the dehydrogenated hydrocarbon stream in line 184 may be fed to a hydrogenation unit to produce the hydrocarbon feed stream in line 102.

Referring back, the first net vapor stream in line 176 is further processed in the purification section 151 to separate hydrogen in a product stream. The first net vapor stream in line 176 is passed to a suction drum 190 to separate liquid from it. In an aspect, the first net vapor stream in line 176 may be combined with a stored hydrogen stream in line 214 to provide a combined net vapor stream in line 179. The combined net vapor stream in line 179 is passed to the suction drum 190 after cooling in a cooler 211. A second net vapor stream is taken in in line 191 and a suction drum liquid stream is taken in line 192 from the suction drum 190. In an aspect, the stored hydrogen stream in line 214 is optional and the first net vapor stream in line 176 may be passed directly to the suction drum 190. The second net vapor stream in line 191 may be compressed in a first product gas compressor 193 to provide a compressed second net vapor stream in line 194. In an embodiment, the second net vapor stream in line 191 may be compressed to a pressure of about 1379 kPa (g) (200 psig) to about 2068 kPa (gauge) (300 psig), preferably about 1586 kPa (g) (230 psig) to about 2068 kPa (g) (300 psig) in the first product gas compressor 193.

The compressed second net vapor stream in line 194 is cooled in a cooler 213 and passed to a discharge drum 195 to separate the liquid from it. A third net vapor stream is taken in line 196 from the discharge drum 195. A discharge drum liquid stream is taken in line 197 from the bottoms of the discharge drum 195. In an embodiment, the discharge drum liquid stream taken in line 197 may be combined with the suction drum liquid stream in line 192 to provide the combined liquid stream in line 198. As explained, the separator liquid stream in line 174 is combined with a combined liquid stream in line 198 to provide a combined liquid dehydrogenated stream in line 175 which is stabilized in the stabilizer column 180. The third net vapor stream in line 196 is compressed in a second product gas compressor 199 to provide a compressed third net vapor stream in line 202. In an exemplary embodiment, the third net vapor stream in line 196 may be compressed to a pressure of about 2758 kPa (g) (400 psig) to about 6895 kPa (g) (1000 psig), preferably about 2758 kPa (g) (400 psig) to around 4137 kPa (g) (600 psig) in the second product gas compressor 199.

Although the embodiment as shown in FIG. 1 the purification section 151 includes two stages of compressors, the first product gas compressor 193 and the second product gas compressor 199, there may be more or less than two stages of compressors. In an aspect, the purification section 151 may comprise between about one and five stages of compressors.

The compressed third net vapor stream in line 202 is cooled in a cooler 215 and passed to a discharge knock out drum (KOD) 216 to separate any liquid from the hydrogen. A KOD liquid stream is taken in line 219 from the discharge KOD 216. In an exemplary embodiment, the KOD liquid stream in line 219 may be combined with the discharge drum liquid stream tin line 197 and the suction drum liquid stream in line 192 to provide the combined liquid stream in line 198. A KOD vapor stream comprising hydrogen is taken in line 218 from the discharge KOD 216. The entirety of the KOD vapor stream in line 218 or a portion of it is taken in a hydrogen product stream and supplied to a downstream process according to the demand for hydrogen by the downstream process. The downstream demand for hydrogen may vary depending on various factors such as low production or turndown or peaking demand of the downstream unit. The dehydrogenation cycle depicted in FIG. 1 must address the swing demand of the hydrogen, which may include varying the feed to process 101 in accordance with the hydrogen demand to the hydrogen header from the downstream unit.

The downstream demand of hydrogen impacts the capacity of the dehydrogenation section 109 for the feed. The feed to the dehydrogenation section 109 is controlled to avoid any non-selective reactions and keep a consistent temperature profile across all of the dehydrogenation reactors. The present process 101 employs the hydrogen header pressure controller 201 to control the production and supply of hydrogen from the dehydrogenation section 109.

A hydrogen product stream is taken in a hydrogen feed line 204 from the KOD vapor stream in line 218. A flow indicator 45 is provided on the hydrogen feed line 204. In an embodiment, a storage hydrogen stream is taken in a storage hydrogen line 203 from the KOD vapor stream in line 218.

In an exemplary embodiment, the hydrogen header pressure controller 201 comprises a primary pressure controller 46 in communication with the hydrogen feed line 204 and the feed line 102. In an aspect, the primary pressure controller 46 performs measuring and comparing steps for the outlet pressure of the hydrogen product stream. The primary pressure controller 46 measures an outlet pressure of the hydrogen product stream in the hydrogen feed line 204 and compares outlet pressure of the hydrogen product stream with a set point pressure range. The set point range of the pressure in the hydrogen feed line 204 defines a normal pressure range for a normal demand of hydrogen from the hydrogen product stream. The primary pressure controller 46 takes no action when the measured outlet pressure of the hydrogen product stream in the hydrogen feed line 204 is within the set point pressure range. Depending on the downstream demand of hydrogen, the pressure of the hydrogen product stream in the hydrogen feed line 204 varies. So, the primary pressure controller 46 takes corrective action when the measured outlet pressure of the hydrogen product stream in the hydrogen feed line 204 is higher or lower than set point pressure range. In an embodiment, the primary pressure controller 46 is configured to adjust a flow rate of the hydrocarbon feed stream to the dehydrogenation reactor section 131 in the hydrocarbon feed line 102 based on a comparison of the outlet pressure of the hydrogen product stream in the hydrogen feed line 204 with the set point pressure range. In an exemplary embodiment, the primary pressure controller 46 is a primary pressure indicating controller (PIC).

The hydrogen header pressure controller 201 comprises a flow indicating controller (FIC) 42 in communication with the hydrocarbon feed line 102. The FIC 42 is in signal communication with a flow indicator (FI) 43 and a control valve 44 both in fluid communication with the hydrocarbon feed line 102. The primary pressure controller 46 is in communication with the FIC 42 to adjust a flow rate of the hydrocarbon feed stream in the hydrocarbon feed line 102 to the dehydrogenation reactor 131 based on a comparison of the outlet pressure of the hydrogen product stream in the hydrogen feed line 204 with the set point pressure range. If the outlet pressure of the hydrogen product stream in the hydrogen feed line 204 is higher than the high set point pressure, this indicates the pressure in the hydrogen feed line 204 is increasing because the hydrogen demand in the downstream hydrogen consuming unit is below the normal range. Consequently, the primary pressure controller 46 sends a signal to the FIC 42 to decrease the flow rate of the hydrocarbon feed stream in the hydrocarbon feed line 102 to the dehydrogenation reactor section 131 to produce less hydrogen. The FIC 42 decreases the flow rate of the hydrocarbon feed stream in the hydrocarbon feed line 102 accordingly by sending a signal to close the control valve 44 from a more open position to a less open position.

Similarly, if the outlet pressure of the hydrogen product stream in the hydrogen feed line 204 is lower than the low set point pressure, this indicates the pressure in the hydrogen feed line 204 is decreasing because the hydrogen demand is above the normal range. Consequently, the primary pressure controller 46 sends a signal to the FIC 42 to increase the flow rate of the hydrocarbon feed stream in the hydrocarbon feed line 102 to the dehydrogenation reactor 131 to produce more hydrogen for meeting the demand. The FIC 42 increases the flow rate of the hydrocarbon feed stream in the hydrocarbon feed line 102 by sending a signal to open the control valve 44 from a less open position to a more open position to increase the flow rate of feed to the dehydrogenation reactor 131.

When the outlet pressure of the hydrogen product stream in the hydrogen feed line 204 decreases quickly, it may lead the hydrogen header pressure controller 201 to change the flow rate of the hydrocarbon feed stream in the hydrocarbon feed line 102 too rapidly which could cause undesirable temperature swings in the hydrogenation reactor 131. The hydrogen header pressure controller 201 comprises a primary controller output rate limiter 57 to limit the rate of change of the output of the primary pressure controller 46. The primary controller output rate limiter 57 is in signal communication with the primary pressure controller 46 and the FIC 42. The output signal of the primary pressure controller 46 is passed to the primary controller output rate limiter 57 and the output signal from the primary controller output rate limiter 57 is passed to the FIC 42. The primary controller output rate limiter 57 limits the rate of change of the output to not exceed a selected set point. If the rate of change of feed rate would exceed the selected set point, the output rate limiter 57 limits the change in the set point of FIC 42 which signals the control valve 44 to open or close accordingly to adjust the flow rate of the hydrocarbon feed stream in the hydrocarbon feed line 102.

In an embodiment, the hydrogen header pressure controller 201 comprises a hydrogen storage drum 210 to store hydrogen and maintain hydrogen pressure during large fluctuations in the outlet pressure of the hydrogen product stream in the hydrogen feed line 204 based on the fluctuations in the hydrogen demand. The hydrogen storage drum 210 stores hydrogen when the demand is decreasing and discharges hydrogen to the hydrogen feed line 204 when demand is increasing as compared to a set point pressure of the hydrogen product stream in the hydrogen feed line 204.

The storage hydrogen stream in the storage hydrogen line 203 is taken and passed to the hydrogen storage drum 210 for storage. A vent hydrogen stream may be taken in a vent line 205 from the storage hydrogen stream. A control valve 49 is provided on the vent line 205. The secondary pressure controller 47 is in communication with the control valve 49 on the vent line 205 to control a vent hydrogen stream in the vent line 205. The remainder of the storage hydrogen stream is taken in line 206 and passed to the hydrogen storage drum 210. A storage inlet line 207 supplies the storage hydrogen stream to the hydrogen storage drum 210. A control valve 52 is provided on the storage inlet line 207. A stored hydrogen stream may be taken from the hydrogen storage drum 210 in a storage discharge line 212. A control valve 55 is provided on the storage discharge line 212.

The hydrogen header pressure controller 201 comprises a secondary pressure controller 47 in communication with the hydrogen feed line 204. In an exemplary embodiment, the secondary pressure controller 47 is a pressure indicating controller (PIC). In an embodiment, the hydrogen header pressure controller 201 comprises a first high selector switch (HSS) 51. The secondary pressure controller 47 controls the feed of the storage hydrogen stream in line 206 to the hydrogen storage drum 210. When the outlet pressure of the hydrogen product stream in the hydrogen feed line 204 is higher than the set point pressure range of the secondary pressure controller 47, the pressure in the hydrogen feed line 204 is increasing because the hydrogen demand is below normal. In this situation, the secondary pressure controller 47 sends a signal to open the control valve 52 on the storage inlet line 207 to send the storage hydrogen stream in the storage hydrogen line 206 to the hydrogen storage drum 210 for storage.

In an exemplary embodiment, the secondary pressure controller 47 has a set point pressure range that has a higher upper set point than the primary pressure controller 46. Consequently, when the pressure in the line 204 increases due to decreased downstream demand, normally the response is to decrease the flowrate in line 102 through control valve 44. However, if the pressure in the line 204 continues to increase due to further decreased demand, the pressure set point on the secondary pressure controller 47 will be exceeded.

If the pressure in line 204 is ever measured by the secondary pressure controller 47 to exceed a maximum set point, the secondary pressure controller 47 may open the control valve 49 more on the vent line 205 from a fully closed or less open position to release the excess hydrogen to a safety relief system or send it to some other location. In an aspect, the output of the secondary pressure controller 47 first opens the control valve 52 on the storage inlet line 207 to the hydrogen storage drum 210 and then opens the control valve 49 on the vent line 205 if the pressure of the secondary pressure controller 47 is above the set point.

The hydrogen header pressure controller 201 comprises a tertiary pressure controller 48 in communication with the hydrogen feed line 204. In another exemplary embodiment, the tertiary pressure controller 48 is a pressure indicator controller (PIC). The tertiary pressure controller 48 controls the withdrawal of stored hydrogen from the hydrogen storage drum 210 and its supply to the hydrogen feed line 204 when hydrogen demand increases. The stored hydrogen stream in line 212 may be discharged to the hydrogen product stream in line 204 when the outlet pressure of the tertiary pressure controller 48 is less than a set point pressure. In this situation, the tertiary pressure controller 48 sends a signal to open the control valve 55 on the storage discharge line 212 to send the stored hydrogen to the hydrogen feed line 204.

In yet another exemplary embodiment, the tertiary pressure controller 48 has a lower set point pressure than the primary pressure controller 46. Consequently, when the pressure in the hydrogen feed line 204 decreases due to increased downstream demand, normally the response is to increase the flowrate in line 102 through the control valve 44. However, if the pressure in the line 204 continues to decrease due to further increased demand, it will diminish to under the set point pressure range on the tertiary pressure controller 48.

The hydrogen header pressure controller 201 controls and maintains the pressure of the hydrogen storage drum 210 in order to maintain an appropriate inventory of stored hydrogen. The hydrogen header pressure controller 201 comprises a hydrogen storage pressure controller 53 on the hydrogen storage drum 210. In an exemplary embodiment, the hydrogen storage pressure controller 53 is a pressure indicating controller (PIC). The hydrogen storage pressure controller 53 compares the pressure of the hydrogen storage drum 210 to a setpoint. When the hydrogen storage drum 210 pressure increases above the setpoint, the hydrogen storage controller 53 first sends a signal to close the control valve 52 on the storage inlet line 207 and then sends a signal to open control valve 55 on the storage discharge line 212. When the hydrogen storage drum 210 pressure decreases below the setpoint, the hydrogen storage controller 53 first sends a signal to close the control valve 55 on the storage discharge line 212 and then sends a signal to open control valve 52 on the storage inlet line 207

In accordance with the present disclosure, the hydrogen header pressure controller 201 comprises a first high selector switch (HSS) 51 in communication with the secondary pressure controller 47, the hydrogen storage pressure controller 53, and the control valve 52 on the storage inlet line 207. The first HSS 51 will select the highest signal from either the secondary pressure controller 47 or the hydrogen storage pressure controller 53 and discard the other signal. In accordance with the present disclosure, the hydrogen header pressure controller 201 comprises a second high selector switch (HSS) 54 in communication with the tertiary pressure controller 48, the hydrogen storage pressure controller 53, and the control valve 55 on the storage discharge line 212. The second HSS 54 will select the highest signal from either the tertiary pressure controller 48 or the hydrogen storage pressure controller 53 and discard the other signal. So, in accordance with this disclosure, when the hydrogen supply meets hydrogen demand, the primary pressure controller 46 will control the pressure via communication with FIC 42 and the secondary pressure controller 47 and tertiary pressure controller 48 will send signals to close control valves 52 and 55, respectively. The hydrogen storage pressure controller 53 will control the pressure in the hydrogen storage drum 210 using control valves 52 and 55 as required. During times when there is a large surplus or deficit of hydrogen, the secondary pressure controller 47 or the tertiary pressure controller 48 will override the signal from the hydrogen storage pressure controller 53 in order to store excess hydrogen or discharge hydrogen from storage.

The hydrogen header pressure controller 201 may comprise a hydrogen inventory calculator 56 which is in communication with the hydrogen storage pressure controller 53, the primary pressure controller 46, and the primary controller output rate limiter 57. The hydrogen inventory calculator 56 may calculate a setpoint for the hydrogen storage pressure controller 53 according to the output signal from the primary pressure controller 46 using a programmed function. In an exemplary embodiment, the setpoint for the hydrogen storage pressure controller 53 is interpolated linearly between a minimum hydrogen storage pressure and a maximum hydrogen storage pressure. In an aspect, the minimum hydrogen storage pressure is about 70 kPa (10 psi) above the pressure of the first net vapor stream in line 176 and the maximum hydrogen storage pressure is about 70 kPa (10 psi) below the pressure of compressed third net vapor stream in line 202. In another embodiment, the programmed function in the hydrogen inventory calculator 56 may include a bias so that the minimum hydrogen storage pressure will occur at an output signal from the primary pressure controller 46 other than the minimum output. In an exemplary embodiment, the minimum setpoint for the hydrogen storage pressure may occur when the output from the primary pressure controller is at about 20% to about 50%.

In an embodiment, the hydrogen inventory calculator 56 is also in communication with the primary controller output rate limiter 57. When the primary controller output rate limiter 57 is active and is limiting the rate of change of the setpoint to FIC 42, it activates a signal which alerts the hydrogen storage pressure controller 53 and the hydrogen inventory calculator 56 to suspend their operations and, in the case of hydrogen storage pressure controller 53, send control signal outputs to control valves 52 and 55 to close. This will prevent the action of the hydrogen storage pressure controller 53 from interfering with the actions of the secondary pressure controller 47 or the tertiary pressure controller 48.

In an aspect, the primary controller output rate limiter 57 may be in communication with a WABT setpoint calculator 58. In an exemplary embodiment, the rate-limited output from the primary pressure controller 46, which is sent to FIC 42, is also sent to the WABT setpoint calculator 58. The function of the WABT setpoint calculator is described below in more detail.

In accordance with the present disclosure, the process 101 comprises a dehydrogenation unit temperature controller 141. In response to the variation in the outlet pressure of the hydrogen product stream in the hydrogen feed line 204, the dehydrogenation unit temperature controller 141 controls the operating temperature of the dehydrogenation reactors 130-160 to keep a consistent temperature profile across all four reactors of the dehydrogenation reactor section 131. Compared to a typical inlet temperature control, the dehydrogenation unit temperature controller 141 controls a weighted average bed temperature (WABT) for each dehydrogenation reactor of the dehydrogenation reactor section 131. The dehydrogenation unit temperature controller 141 comprises a WABT controller to control the temperatures of each of the dehydrogenation reactors of the dehydrogenation reactor section 131 rather than an inlet temperature controller. The WABT controllers control an average reactor temperature rather than only an inlet temperature. The average reactor temperature may be defined as an average of the inlet and outlet temperatures of the reactor. Each reactor may have its own WABT calculation with the weighting between inlet and outlet temperature set differently. During turndown, the inlet temperatures for each dehydrogenation reactor will be lower than normal and the outlet temperatures will be higher than normal. By controlling average temperature, the dehydrogenation unit temperature controller 141 controls changes in both inlet and outlet temperature of the dehydrogenation reactor. Turndown impact will be spread across all four dehydrogenation reactors during turndown rather than allowing the bulk of the reaction to occur in just a few upstream dehydrogenation reactors.

The dehydrogenation unit temperature controller 141 comprises a first WABT measuring device 21 for calculating the WABT of the first dehydrogenation reactor 130. In an exemplary embodiment, the weighting for WABT calculation in the first WABT measuring device 21 for the first dehydrogenation reactor 130 is by catalyst weight in the first dehydrogenation reactor 130. The WABT may be calculated using temperature measurements at several locations in the catalyst bed of the first dehydrogenation reactor 130. The first WABT measuring device 21 is in communication with a first inlet temperature indicator (TI) 22 to measure the first inlet temperature and a first outlet temperature indicator (TI) 24 to measure the outlet temperature for the first dehydrogenation reactor 130 and calculate the WABT of the first dehydrogenation reactor 130 based on the measured temperatures. The first WABT measuring device 21 is in communication with a first total heat input controller (TQIC) 25 which adjusts the WABT of the first dehydrogenation reactor 130 to a WABT set point range. The first total heat input controller 25 is in communication with the first feed heater 122 to control the heating of the combined hydrocarbon feed stream in line 121 in the first feed heater 122. The first WABT measuring device 21 sends a signal to the first total heat input controller 25 which compares the WABT of the first dehydrogenation reactor 130 to a WABT set point range. The WABT set point range may comprise a WABT high set point and a WABT low set point. If the measured WABT of the first dehydrogenation reactor 130 is not within the WABT set point range, the first total heat input controller 25 sends a signal to the power controller 71 on the first feed heater 122 to increase or decrease the heating. If the WABT of the first dehydrogenation reactor 130 is higher than the WABT high set point, the first total heat input controller 25 sends a signal to the power controller 71 on the first feed heater 122 to decrease the heating. If the WABT of the first dehydrogenation reactor 130 is lower than the WABT low set point, the first total heat input controller 25 sends a signal to the power controller 71 on the first feed heater 122 to increase the heating.

In an embodiment, the first feed heater 122 may be a fired heater. In such an embodiment, the power controller 71 on the first feed heater 122 would control the supply of fuel to the first feed heater 122 rather than power. So, for a fired heater 122, if the measured WABT of the first dehydrogenation reactor 130 is not within the WABT set point range, the first total heat input controller 25 sends a signal to the power controller 71 on the first feed heater 122 to increase or decrease the supply of fuel. If the WABT of the first dehydrogenation reactor 130 is higher than the WABT high set point, the first total heat input controller 25 sends a signal to the power controller 71 on the first feed heater 122 to decrease heating by decreasing the flow rate of fuel. If the WABT of the first dehydrogenation reactor 130 is lower than the WABT low set point, the first total heat input controller 25 sends a signal to the power controller 71 on the first feed heater 122 to increase the heating by increasing the flow rate of fuel.

In an embodiment, one or both of the first total heat input controller 25 and the power controller 71 may be configured to adjust a heat duty of the first feed heater 122 when the average reactor temperature of the first dehydrogenation reactor 130 deviates from a set point average reactor.

In an aspect, the dehydrogenation unit temperature controller 141 comprises a first temperature indicating controller (TIC) 23 which may take an override control to avoid over-firing of the first feed heater 122 and exceeding the design temperature of the first dehydrogenation reactor 130 or an inlet temperature of the first dehydrogenation reactor 130 exceeds a set point temperature. In an exemplary embodiment, the first temperature indicating controller (TIC) 23 is an override temperature controller. For overriding, the dehydrogenation unit temperature controller 141 comprises a selector switch which is a first low selector switch (LSS) 125 to stop a measured temperature from exceeding a design temperature upper limit. As long as the inlet temperature of the first dehydrogenation reactor 130 is below the design temperature upper limit of the first dehydrogenation reactor 130, the first total heat input controller 25 controls the power controller 71 to adjust the heating from the first feed heater 122. Once the inlet temperature of the first dehydrogenation reactor 130 exceeds the design temperature upper limit of the first dehydrogenation reactor 130, the first TIC 23 overrides first total heat input controller 25 through the selector switch, the first LSS 125 and controls the power controller 71 to adjust the heating from the first feed heater 122. The override prevents the inlet temperature of the first dehydrogenation reactor 130 from exceeding the design temperature upper limit of the first dehydrogenation reactor 130. So, when the inlet temperature of the first dehydrogenation reactor 130 exceeds the design temperature upper limit of the first dehydrogenation reactor 130, the first LSS 125 selects the output signal from the first TIC 23 and discards the output signal from the first total heat input controller 25 to provide override control to the first TIC 23 for controlling the power controller 71 to adjust the heating from the first feed heater 122.

Similarly, the dehydrogenation unit temperature controller 141 comprises a second WABT measuring device 26 for calculating the WABT of the second dehydrogenation reactor 140. In an exemplary embodiment, the weighting for WABT calculation in the second WABT measuring device 26 for the second dehydrogenation reactor 140 is by catalyst weight in the second dehydrogenation reactor 140. The WABT may be calculated using temperature measurements at several locations in the catalyst bed of the second dehydrogenation reactor 140. The second WABT measuring device 26 is in communication with a second inlet TI 27 to measure the inlet temperature and a second outlet TI 61 to measure the outlet temperature for the second dehydrogenation reactor 140 and calculate the WABT of the second dehydrogenation reactor 140 based on the measured temperatures. The second WABT measuring device 26 is in communication with a second total heat input controller 29 which controls or adjusts the WABT of the second dehydrogenation reactor 140. The second total heat input controller 29 is in communication with the second feed heater 133 to control the heating of the first dehydrogenated effluent stream in line 132 in the second feed heater 133. The second WABT measuring device 26 sends a signal to the second total heat input controller 29 which compares the WABT of the second dehydrogenation reactor 140 to a WABT set point range. If the measured WABT of the second dehydrogenation reactor 140 is not within the WABT set point range, the second total heat input controller 29 sends a signal to the power controller 72 on the second feed heater 133 to increase or decrease the heating. If the WABT of the second dehydrogenation reactor 140 is higher than the WABT high set point, the second total heat input controller 29 sends a signal to the power controller 72 on the second feed heater 133 to decrease the heating. If the WABT of the second dehydrogenation reactor 140 is lower than the WABT low set point, the second total heat input controller 29 sends a signal to the power controller 72 on the second feed heater 133 to increase the heating.

In an embodiment, the second feed heater 133 may be a fired heater. In such an embodiment, the power controller 72 on the second feed heater 133 would control the supply of fuel to the second feed heater 133 rather than power. So, for a fired heater 133, if the measured WABT of the second dehydrogenation reactor 140 is not within the WABT set point range, the second total heat input controller 29 sends a signal to the power controller 72 on the second feed heater 133 to increase or decrease the flow rate of fuel. If the WABT of the second dehydrogenation reactor 140 is higher than the WABT high set point, the second total heat input controller 29 sends a signal to the power controller 72 on the second feed heater 133 to decrease the heating by decreasing the flow rate of fuel. If the WABT of the second dehydrogenation reactor 140 is lower than the WABT low set point, the second total heat input controller 29 sends a signal to the power controller 72 on the second feed heater 133 to increase the heating by increasing the flow rate of fuel.

In an embodiment, one or both of the second total heat input controller 29 and the power controller 72 may be configured to adjust a heat duty of the second feed heater 133 when the average reactor temperature of the second dehydrogenation reactor 140 deviates from a set point average reactor.

In an aspect, the dehydrogenation unit temperature controller 141 comprises a second TIC 28 which may take an override control to avoid over-firing of the second feed heater 133 and exceeding the design temperature of the second dehydrogenation reactor 140 or an inlet temperature of the second dehydrogenation reactor 140 exceeds a set point temperature. In an exemplary embodiment, the second TIC 28 is an override temperature controller. For overriding, the dehydrogenation unit temperature controller 141 comprises a selector switch which is a second LSS 135. When the inlet temperature of the second dehydrogenation reactor 140 is below the design temperature of the second dehydrogenation reactor 140, the second total heat input controller 29 controls the power controller 72 to adjust the heating from the second feed heater 133. Once the inlet temperature of the second dehydrogenation reactor 140 exceeds the design temperature of the second dehydrogenation reactor 140, the second LSS 135 provides override control to the second TIC 28 from the second total heat input controller 29. Now, the second TIC 28 controls the power controller 72 to adjust the heating from the second feed heater 133 so that the inlet temperature of the second dehydrogenation reactor 140 does not exceed the design temperature of the second dehydrogenation reactor 140.

For the third dehydrogenation reactor 150, the dehydrogenation unit temperature controller 141 comprises a third WABT measuring device 32 for calculating the WABT of the third dehydrogenation reactor 150. In an exemplary embodiment, the weighting for WABT calculation in the third WABT measuring device 32 for the third dehydrogenation reactor 150 is by catalyst weight in the third dehydrogenation reactor 150. The WABT may be calculated using temperature measurements at several locations in the catalyst bed of the third dehydrogenation reactor 150. The third WABT measuring device 32 is in communication with a third inlet TI 31 to measure the inlet temperature and a third outlet TI 33 to measure the outlet temperature for the third dehydrogenation reactor 150 and calculate the WABT of the third dehydrogenation reactor 150 based on the measured temperatures. The third WABT measuring device 32 is in communication with a third total heat input controller 35 which controls or adjusts the WABT of the third dehydrogenation reactor 150. The third total heat input controller 35 is in communication with the third feed heater 143 to control the heating of the second dehydrogenated effluent stream in line 142 in the third feed heater 143. The third WABT measuring device 32 sends a signal to the third total heat input controller 35 which compares the WABT of the third dehydrogenation reactor 150 to a set WABT range. If the measured WABT of the third dehydrogenation reactor 150 is not within the WABT set point range, the third total heat input controller 35 sends a signal to the power controller 73 on the third feed heater 143 to increase or decrease the heating. If the WABT of the third dehydrogenation reactor 150 is higher than the WABT high set point, the third total heat input controller 35 sends a signal to the power controller 73 on the third feed heater 143 to decrease the heating. If the WABT of the third dehydrogenation reactor 150 is lower than the WABT low set point, the third total heat input controller 35 sends a signal to the power controller 73 on the third feed heater 143 to increase the heating.

In an embodiment, the third feed heater 143 may be a fired heater. In such an embodiment, the power controller 73 on the third feed heater 143 would control the supply of fuel to the third feed heater 143 rather than power. So, for a fired heater 143, if the measured WABT of the third dehydrogenation reactor 150 is not within the WABT set point range, the third total heat input controller 35 sends a signal to the power controller 73 on the third feed heater 143 to increase or decrease the flow rate of fuel. If the WABT of the third dehydrogenation reactor 150 is higher than the WABT high set point, the third total heat input controller 35 sends a signal to the power controller 73 on the third feed heater 143 to decrease the heating by decreasing the flow rate of fuel. If the WABT of the third dehydrogenation reactor 150 is lower than the WABT low set point, the third total heat input controller 35 sends a signal to the power controller 73 on the third feed heater 143 to increase the heating by increasing the flow rate of fuel.

In an embodiment, one or both of the third total heat input controller 35 and the power controller 73 may be configured to adjust a heat duty of the third feed heater 143 when the average reactor temperature of the third dehydrogenation reactor 150 deviates from a set point average reactor.

In an aspect, the dehydrogenation unit temperature controller 141 comprises a third TIC 34 which may take an override control to avoid over-firing of the third feed heater 143 and exceeding the design temperature of the third dehydrogenation reactor 150 or an inlet temperature of the third dehydrogenation reactor 150 exceeds a set point temperature. In an exemplary embodiment, the third TIC 34 is an override temperature controller. For overriding, the dehydrogenation unit temperature controller 141 comprises a selector switch which is a third LSS 145. When the inlet temperature of the third dehydrogenation reactor 150 is below the design temperature of the third dehydrogenation reactor 150, the third total heat input controller 35 controls the power controller 73 to adjust the heating from the third feed heater 143. Once the inlet temperature of the third dehydrogenation reactor 150 exceeds the design temperature of the third dehydrogenation reactor 150, the third LSS 145 provides override control to the third TIC 34 from the third total heat input controller 35. Now, the third TIC 34 controls the power controller 73 to adjust the heating from the third feed heater 143 so that the inlet temperature of the third dehydrogenation reactor 150 does not exceed the design temperature of the third dehydrogenation reactor 150.

The dehydrogenation unit temperature controller 141 comprises a fourth WABT measuring device 36 for calculating the WABT of the fourth dehydrogenation reactor 160. In an exemplary embodiment, the weighting for inlet temperature calculation in the fourth WABT measuring device 36 for the fourth dehydrogenation reactor 160 is by catalyst weight in the fourth dehydrogenation reactor 160. The inlet temperature may be calculated using temperature measurements at several locations in the catalyst bed of the fourth dehydrogenation reactor 160. The fourth WABT measuring device 36 is in communication with a fourth inlet TI 37 to measure the inlet temperature and a fourth outlet TI 41 to measure the outlet temperature for the fourth dehydrogenation reactor 160 and calculate the inlet temperature of the fourth dehydrogenation reactor 160 based on the measured temperatures. The fourth WABT measuring device 36 is in communication with a fourth total heat input controller 39 which controls or adjusts the inlet temperature of the fourth dehydrogenation reactor 160. The fourth total heat input controller 39 is in communication with the fourth feed heater 153 to control the heating of the third dehydrogenated effluent stream in line 152 in the fourth feed heater 153. The fourth WABT measuring device 36 sends a signal to the fourth total heat input controller 39 which compares the inlet temperature of the fourth dehydrogenation reactor 160 to a WABT set point range. If the measured WABT of the fourth dehydrogenation reactor 160 is not within the set WABT range, the fourth total heat input controller 39 sends a signal to the power controller 74 on the fourth feed heater 153 to increase or decrease the heating. If the inlet temperature of the fourth dehydrogenation reactor 160 is higher than the WABT high set point, the fourth total heat input controller 39 sends a signal to the power controller 74 on the fourth feed heater 153 to decrease the heating. If the inlet temperature of the fourth dehydrogenation reactor 160 is lower than the WABT low set point, the fourth total heat input controller 39 sends a signal to the power controller 74 on the fourth feed heater 153 to increase the heating.

In an embodiment, the fourth feed heater 153 may be a fired heater. In such an embodiment, the power controller 74 on the fourth feed heater 153 would control the supply of fuel to the fourth feed heater 153 rather than power. So, for a fired heater 153, if the measured WABT of the fourth dehydrogenation reactor 160 is not within the set WABT range, the fourth total heat input controller 39 sends a signal to the power controller 74 on the fourth feed heater 153 to increase or decrease the flow rate of fuel. If the WABT of the fourth dehydrogenation reactor 160 is higher than the WABT high set point, the fourth total heat input controller 39 sends a signal to the power controller 74 on the fourth feed heater 153 to decrease the heating by decreasing the flow rate of fuel. If the WABT of the fourth dehydrogenation reactor 160 is lower than the WABT low set point, the fourth total heat input controller 39 sends a signal to the power controller 74 on the fourth feed heater 153 to increase the heating by increasing the flow rate of fuel.

In an embodiment, one or both of the fourth total heat input controller 39 and the power controller 74 may be configured to adjust a heat duty of the fourth feed heater 153 when the average reactor temperature of the fourth dehydrogenation reactor 160 deviates from a set point average reactor.

The dehydrogenation unit temperature controller 141 may comprise a fourth TIC 38 which may take an override control to avoid over-firing of the fourth feed heater 153 and exceeding the design temperature of the fourth dehydrogenation reactor 160 or an inlet temperature of the fourth dehydrogenation reactor 160 exceeds a set point temperature. In an exemplary embodiment, the fourth TIC 38 is an override temperature controller. For overriding, the dehydrogenation unit temperature controller 141 comprises a selector switch which is a fourth LSS 155. When the inlet temperature of the fourth dehydrogenation reactor 160 is below the design temperature of the fourth dehydrogenation reactor 160, the fourth total heat input controller 39 controls the power controller 74 to adjust the heating from the fourth feed heater 153. Once the inlet temperature of the fourth dehydrogenation reactor 160 exceeds the design temperature of the fourth dehydrogenation reactor 160, the fourth LSS 155 provides override control to the fourth TIC 38 from the fourth total heat input controller 39. Now, the fourth TIC 38 controls the power controller 74 to adjust the heating from the fourth feed heater 153 so that the inlet temperature of the fourth dehydrogenation reactor 160 does not exceed the design temperature of the fourth dehydrogenation reactor 160.

In an aspect, the embodiment as shown in FIG. 1 may include a WABT setpoint calculator 58. The WABT setpoint calculator 58 is in communication with the first total heat input controller 25, the second total heat input controller 29, the third total heat input controller 35, and the fourth total heat input controller 39. The WABT setpoint calculator 58 may take an input from the primary controller output rate limiter 57 and calculates setpoints for the first total heat input controller 25, the second total heat input controller 29, the third total heat input controller 35, and the fourth total heat input controller 39. In another aspect, the WABT setpoint calculator 58 may automatically adjust an average reactor temperature set point of the dehydrogenation reactors 130-160, wherein the average reactor temperature set point is a function of the flow rate of the hydrocarbon feed stream in feed line 102.

A “typical” process is designed to process a given amount of hydrocarbon feed and produce however much hydrogen which comes off of it. In the present process, the typical hydrogen production is improved by allowing the users to determine the hydrogen load they want in the hydrogen users and the process will automatically adjust the hydrocarbon feed rate accordingly. In accordance with the present disclosure, a hydrogen header pressure controller 201 is provided which comprises a primary pressure controller 46 in communication with the hydrogen feed line 204 and the hydrocarbon feed line 102. The primary pressure controller 46 would automatically adjust the hydrocarbon feed rate in the feed line 102.

Further, a “typical” process is designed for modest rates of change to the reactor feeds and temperatures. In the present process, the hydrocarbon feed rate in the feed line 102 is slowly ramp up in order to limit sudden changes to reactor temperatures. When the flow rate of the liquid hydrocarbon feed stream in feed line 102 to the dehydrogenation reactors 130-160 is increased, the temperatures on the outlet of the dehydrogenation reactors 130-160 drop as the reaction is endothermic, which requires more power in the heaters 122, 133, 143, and 153. We typically limit this rate of change to 28° C. (50° F.)/hr, which limits how fast we can ramp up and down the feed rates of hydrocarbon feed stream in feed line 102. However, if this unit is the only supplier of green hydrogen to the hydrogen users, it might have to respond quickly to changes in the hydrogen demand downstream of the process. Thus, the present disclosure provides the hydrogen header pressure controller 201 comprising the primary pressure controller 46 in combination with the secondary pressure controller 47, the tertiary pressure controller 48 and the hydrogen storage pressure controller 53 to smooth demand to allow the process time to keep up.

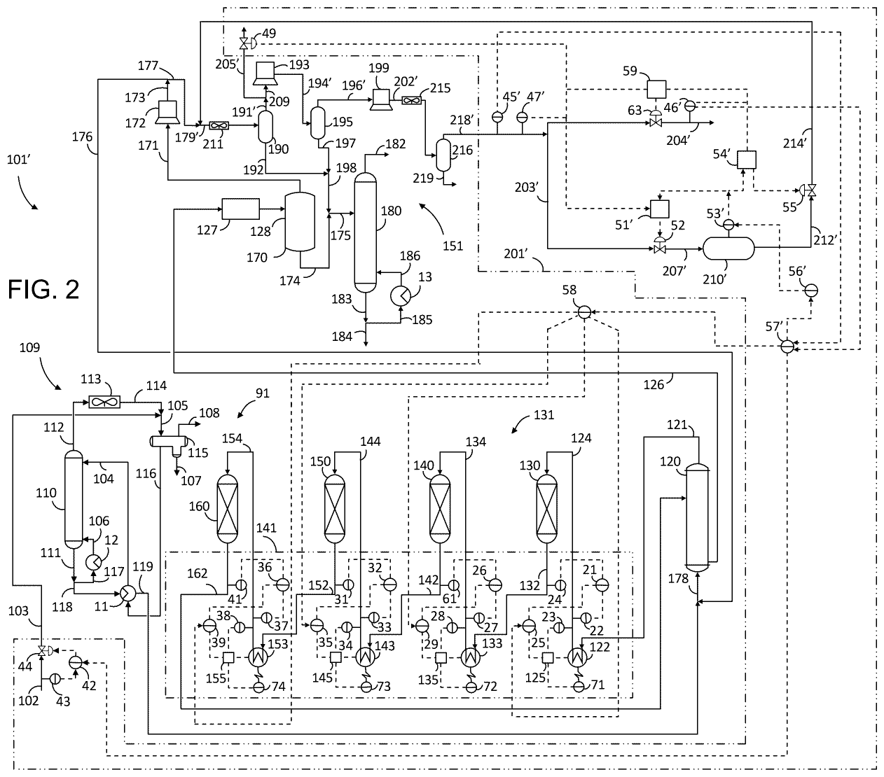

FIG. 2 shows an alternative embodiment of the process of providing a hydrogen stream 101′. The hydrogen header pressure controller 201′ of the process 101′ as shown in FIG. 2 comprises fewer controllers in communication with the hydrogen feed line 204′ as compared to the hydrogen header pressure controller 201 of the process 101 shown in FIG. 1. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

As shown in FIG. 2, a vent hydrogen stream in line 205′ is taken from the second net vapor stream in line 191′. The remainder of the first overhead stream is taken in line 209 which is compressed in the first product gas compressor 193 to provide a compressed second net vapor stream in line 194′. The compressed second net vapor stream in line 194′ is passed to the discharge drum 195. A third net vapor stream is produced in line 196′ from the discharge drum 195. The third net vapor stream in line 196′ is compressed in the second product gas compressor 199 to provide a compressed third net vapor stream in line 202′. The compressed third net vapor stream in line 202′ is cooled in the cooler 215 and passed to the discharge knock out drum (KOD) 216 to separate any liquid from the hydrogen. A KOD liquid stream is taken in line 219 from the discharge KOD 216. A KOD vapor stream comprising hydrogen is taken in line 218′ from the discharge KOD 216. The entirety of the KOD vapor stream in line 218′ or a portion of it may be taken in a hydrogen product stream and supplied to a downstream process as per the hydrogen demand.

In an embodiment, a hydrogen product stream is taken in a hydrogen feed line 204′ from the KOD vapor stream in line 218′. In an embodiment, a storage hydrogen stream is taken in a storage hydrogen line 203′ also from the KOD vapor stream in line 218′.

The hydrogen header pressure controller 201′ in the embodiment as shown in FIG. 2 comprises a primary pressure controller 46′ in communication with the hydrogen feed line 204′ and the feed line 102. A control valve 63 is provided on the hydrogen feed line 204′. The primary pressure controller 46′ is in communication with the control valve 63 to control the flow of the hydrogen product stream though the hydrogen feed line 204′. In an aspect, the primary pressure controller 46′ may limit the time rate of change in flow rate of the hydrocarbon feed stream in the feed line 102 charged to the dehydrogenation reactor section 131 based on the comparison of the hydrogen production flowrate in line 202′ with a set point of the hydrogen production flowrate. In an embodiment, the control valve 63 is a net valve of the hydrogen header pressure controller 201′. The primary pressure controller 46′ measures an outlet pressure of the hydrogen product stream in the hydrogen feed line 204′ and compares the measured outlet pressure of the hydrogen product stream with a set point pressure range for the hydrogen feed line 204′. If the outlet pressure of the hydrogen product stream in the hydrogen feed line 204′ is higher than the set point pressure range, the pressure in the hydrogen feed line 204′ is increasing because the hydrogen demand in the downstream hydrogen consuming unit is below the normal range. In this situation, the primary pressure controller 46′ sends an output signal to close the control valve 63 from a more open position to a less open position to reduce the flow rate of the hydrogen product stream in the hydrogen feed line 204′. If the outlet pressure of the hydrogen product stream in the hydrogen feed line 204′ is lower than the set point pressure range, the pressure in the hydrogen feed line 204′ is decreasing because the hydrogen demand is above normal. In this situation, the primary pressure controller 46′ sends an output signal to open the control valve 63 from a less open position to a more open position to increase the flow rate of the hydrogen product stream in the hydrogen feed line 204′.

In the embodiment as shown in FIG. 2, the primary pressure controller 46′ controls the withdrawal of stored hydrogen from the hydrogen storage drum 210′ and its supply to the hydrogen feed line 204′ when hydrogen demand increases. So, when the pressure in the hydrogen feed line 204′ decreases due to increased downstream demand, normally the response is to increase the flowrate in line 102 through the control valve 44. However, if the pressure in the line 204′ continues to decrease due to further increased demand, it will diminish below a pressure set point of the primary pressure controller 46′, resulting in opening of the control valve 55 to discharge hydrogen from the storage tank 210′ in line 212′.

An FI 45′ is provided on the compressed third net vapor stream in line 202′. The primary controller output rate limiter 57′ is in communication with the primary pressure controller 46′, the FIC 42, and the FI 45′.

As shown, the secondary pressure controller 47′ is in communication with the compressed third net vapor stream in line 202′. The secondary pressure controller 47′ controls the withdrawal of the storage hydrogen stream in the storage hydrogen line 203′ and controls the feed of the storage hydrogen stream to the hydrogen storage drum 210′. If the outlet pressure of the hydrogen product stream in the hydrogen feed line 204′ is higher than the set point pressure range, the pressure in the hydrogen feed line 204′ increases because the hydrogen demand in the downstream hydrogen consuming unit is below the normal range. In this situation, the secondary pressure controller 47′ sends an output signal to open the control valve 52 on the storage inlet line 207′ from a less open position to a more open position to pass the storage hydrogen stream to the hydrogen storage drum 210′ for storage. Further, the secondary pressure controller 47′ may open the control valve 49 on the vent line 205′ from a fully closed or less open position to a more open position to release the excess hydrogen flow to a downstream location or even to a safety relief system when the pressure in the 202′ exceeds a maximum set point pressure.

If the outlet pressure of the hydrogen product stream in the hydrogen feed line 204′ is lower than the set point pressure range, the pressure in the hydrogen feed line 204′ decreases because the hydrogen demand in the downstream hydrogen consuming unit is above the normal range. In this situation, the secondary pressure controller 47′ sends an output signal to move the control valve 52 on the storage inlet line 207′ from a more open position to a more closed position to stop or reduce the flow of the storage hydrogen stream to the hydrogen storage drum 210′.

The hydrogen header pressure controller 201′ comprises a LSS 59 in communication with the primary pressure controller 46′ and the secondary pressure controller 47′. The LSS 59 prevents the outlet pressure of the hydrogen product stream in the hydrogen feed line 204′ from going below the low set point pressure of the set point range. When the outlet pressure of the hydrogen product stream in the hydrogen feed line 204′ reaches the low set point pressure, the LSS 59 may provide override control of the control valve 49 on the vent line to the primary pressure controller 46′ from the secondary pressure controller 47′. In override control, the LSS 59 selects the output signal from the primary pressure controller 46′ and discards the output signal from the secondary pressure controller 47′. The primary pressure controller 46′ sends an output signal to close the control valve 49 on the vent line to stop the release of the vent hydrogen stream.

When the outlet pressure in the hydrogen feed line 204′ is higher than the pressure set point range, the primary pressure controller 46′ sends an output signal to the FIC 42 to reduce the flow rate of the hydrocarbon feed stream in the hydrocarbon feed line 102. Similarly, when the outlet pressure in the hydrogen feed line 204′ is lower than the set point pressure range, the primary pressure controller 46′ sends an output signal to the FIC 42 to increase the flow rate of the hydrocarbon feed stream in the hydrocarbon feed line 102.

The hydrogen header pressure controller 201′ comprises a first high selector switch (HSS) 51′ and a second high selector switch (HSS) 54′ to provide override control to the dynamic PIC 53′ and stop the pressure in the hydrogen storage drum 210′ from exceeding a maximum limit. The first HSS 51′ is in communication with the secondary pressure controller 47′, the dynamic PIC 53′ on the hydrogen storage drum 210′, and the control valve 52 on the storage inlet line 207′. The second HSS 54′ is in communication with the primary pressure controller 46′, the dynamic PIC 53′, and the control valve 55 on the storage discharge line 212′. Once the pressure in the hydrogen storage drum 210′ reaches the maximum pressure set point, the dynamic PIC 53′ on the hydrogen storage drum 210′ overrides the secondary pressure controller 47′ through the selector switch of the first HSS 51′ and controls the control valve 52 on the storage inlet line 207′. So, when the pressure in the hydrogen storage drum 210′ reaches the maximum pressure set point, the first HSS 51′ selects the output signal from the dynamic PIC 53′ and discards the output signal from the secondary pressure controller 47′ to provide override control to the dynamic PIC 53′ for controlling the valve 52. The dynamic PIC 53′ sends an output signal to close the control valve 52 on the storage inlet line 207′ when the pressure in the hydrogen storage drum 210′ reaches the maximum pressure set point. Further, the dynamic PIC 53′ on the hydrogen storage drum 210′ overrides the primary pressure controller 46′ through a selector switch, the second HSS 54′ and controls the control valve 55 on the storage discharge line 212′. So, when the pressure in the hydrogen storage drum 210′ reaches the maximum pressure set point, the second HSS 54′ selects the output signal from the dynamic PIC 53′ and discards the output signal from the primary pressure controller 46′ to provide override control to the dynamic PIC 53′ for controlling the valve 55. The PIC 53′ sends an output signal to open the control valve 55 more on the storage discharge line 212′ when the pressure in the hydrogen storage drum 210′ reaches the high pressure set point to discharge the stored hydrogen from the hydrogen storage drum 210′. A controlled stored hydrogen stream is discharged in a controlled storage discharge line 214′. The controlled stored hydrogen stream in line 214′ is recycled back to the purification section 151. The controlled stored hydrogen stream in line 214′ is combined with the recycled vapor stream in line 177 to provide the combined net vapor stream in line 179′. The controlled stored hydrogen stream in line 214′ is taken in the hydrogen feed line 204′ through the second net vapor in line 191′ and the third net vapor stream in line 196′.

Further, the hydrogen header pressure controller 201′ may comprise an FI 45′ in communication with the compressed overhead vapor stream in line 202′. The FI 45′ sends an output signal of a flow rate of the compressed overhead vapor stream in line 202′ to the primary controller output rate limiter 57′. Rate Limiter 57′ adjusts the output based on input from FI 45′ and will pass an increase in setpoint request to dynamic setpoint controller 56′ when the feed rate in line 202′ descends below a feed rate set point range to store more hydrogen. When FI 45′ measures a flowrate in line 202′ above the feed rate set point range, the rate limiter 57′ will pass a decrease in setpoint request to the dynamic setpoint controller 56′ to store less hydrogen and return hydrogen to the system in line 214′ as the pressure is reduced at PIC 53′pressure controller on storage tank 210′.

### Example

A comparative study was performed to compare a prior art process with the present process of providing a hydrogen stream including the hydrogen header pressure controller. Total reactor temperature differential for all four reactors in the study was 884° F. Results of the study are provided in the Table below:

TABLE

| | | | Example with hydrogen | |
|---|---|---|---|---|
| | Prior art | | header pressure controller | |
| Hydrogen Production Rate | 6,300 kg/h (13,890 lb/h) | | | |
| Temperature rate of change limit | 56° C./hr (100° F./hr) | | | |
| Hydrogenated hydrocarbon feed rate | 11% design feed/hr | | 80% design feed/hr | |
| of change limit | | | | |
| Required hydrogen storage | 4.4 hr design feed | | 0.6 hr design feed | |
| Required hydrogen storage mass | 27,720 kg | (61,100 lb) | 3,780 kg | (8,330 lb) |
| Required hydrogen storage volume | 13,200 m³ | (466,000 ft³) | 1,800 m³ | (63,600 ft³) |
| Required hydrogen storage vessels | 4 | | 1 | |
| Hydrogen storage vessel dimensions | 353 | m² | 221 | m² |
| (D × L) | | | | |
| Hydrogen storage vessel cost | US$5.3 | MM | US$3.4 | MM |
| (equipment only) per vessel | | | | |
| Hydrogen storage vessel cost | US$21.3 | MM | US$3.4 | MM |
| (equipment only) | | | | |

Conventionally, marginal change in feed rate affected last reactor temperature differential rate of change, so all change in feed is proportional to the change in the last reactor temperature differential. With the present process, the overall WABT was controlled, which means both inlet and outlet temperatures will be adjusted with changes in feed rate. This spreads the change to eight different points, which allows the feed rate to be adjusted eight times faster.

### Specific Embodiments

While the following is described in conjunction with specific embodiments, it will be understood that this description is intended to illustrate and not limit the scope of the preceding description and the appended claims.

A first embodiment of the present disclosure is a process of providing a hydrogen stream, comprising charging a hydrocarbon feed stream to a dehydrogenation reactor to provide a dehydrogenated stream; separating the dehydrogenated stream in a separator to provide a vapor stream comprising hydrogen and a liquid stream; taking a hydrogen product stream from the vapor stream; comparing an outlet pressure of the hydrogen product stream in a hydrogen feed line with a set point pressure; and adjusting a flow rate of the hydrocarbon feed stream to the dehydrogenation reactor based on a comparison of the outlet pressure with the set point pressure. An embodiment of the present disclosure is one, any or all of prior embodiments in this paragraph up through the first embodiment in this paragraph further comprising limiting the time rate of change in flow rate of the hydrocarbon feed stream charged to the dehydrogenation reactor. An embodiment of the present disclosure is one, any or all of prior embodiments in this paragraph up through the first embodiment in this paragraph further comprising taking a storage hydrogen stream from the vapor stream; passing the storage hydrogen stream to a storage drum when the outlet pressure exceeds the set point pressure; and discharging a stored hydrogen stream from the storage drum to the hydrogen product stream when the outlet pressure is less than the set point pressure. An embodiment of the present disclosure is one, any or all of prior embodiments in this paragraph up through the first embodiment in this paragraph, wherein a secondary pressure controller measures the outlet pressure in the hydrogen feed line and admits the storage hydrogen stream to the storage drum when the outlet pressure exceeds the set point pressure, and wherein the secondary pressure controller has a higher set point than the primary pressure controller. An embodiment of the present disclosure is one, any or all of prior embodiments in this paragraph up through the first embodiment in this paragraph, wherein a tertiary pressure controller measures the outlet pressure in the hydrogen feed line and discharges the stored hydrogen stream from the storage drum when the outlet pressure is inferior to the set point pressure. An embodiment of the present disclosure is one, any or all of prior embodiments in this paragraph up through the first embodiment in this paragraph, wherein the tertiary pressure controller has a lower set point than the primary pressure controller. An embodiment of the present disclosure is one, any or all of prior embodiments in this paragraph up through the first embodiment in this paragraph, wherein the secondary pressure controller 47 is in communication with a vent line to release a vent hydrogen stream when the outlet pressure of the storage drum exceeds the set point pressure. An embodiment of the present disclosure is one, any or all of prior embodiments in this paragraph up through the first embodiment in this paragraph further comprising compressing the vapor stream in a recycle gas compressor to provide a compressed vapor stream; taking the vent hydrogen stream in the vent line from the compressed vapor stream; and taking the hydrogen product stream from the compressed vapor stream An embodiment of the present disclosure is one, any or all of prior embodiments in this paragraph up through the first embodiment in this paragraph, wherein the vent hydrogen stream is taken from the vapor stream before compressing the vapor stream. An embodiment of the present disclosure is one, any or all of prior embodiments in this paragraph up through the first embodiment in this paragraph further comprising heating the hydrocarbon feed stream in a heater to provide a heated hydrocarbon feed stream; passing the heated hydrocarbon feed stream to the dehydrogenation reactor; measuring an inlet temperature of the heated hydrocarbon feed stream to the reactor and an outlet temperature of the dehydrogenated stream from the reactor; calculating an average reactor temperature from a measured inlet temperature of the heated hydrocarbon feed stream and a measured outlet temperature of the dehydrogenated stream; and adjusting a duty of the heater when the average reactor temperature deviates from a set point average reactor temperature. An embodiment of the present disclosure is one, any or all of prior embodiments in this paragraph up through the first embodiment in this paragraph further comprising automatically adjusting an average reactor temperature set point, wherein the average reactor temperature set point is a function of the flow rate of the hydrocarbon feed stream. An embodiment of the present disclosure is one, any or all of prior embodiments in this paragraph up through the first embodiment in this paragraph further comprising a low selector switch in communication with the heater; and an override temperature controller in communication with the low selector switch, the override temperature controller overrides a temperature controller when a reactor inlet temperature exceeds a set point temperature. An embodiment of the present disclosure is one, any or all of prior embodiments in this paragraph up through the first embodiment in this paragraph further comprising compressing at least a portion of the vapor stream in a compressor to provide the hydrogen product stream; and discharging the stored hydrogen stream to an inlet of the compressor. An embodiment of the present disclosure is one, any or all of prior embodiments in this paragraph up through the first embodiment in this paragraph further comprising comparing a pressure of the storage drum to a hydrogen storage set point pressure; passing the storage hydrogen stream to the storage drum when the storage drum pressure is below a hydrogen storage set point pressure; and discharging the stored hydrogen stream from the storage drum when the storage drum pressure exceeds a hydrogen storage set point pressure. An embodiment of the present disclosure is one, any or all of prior embodiments in this paragraph up through the first embodiment in this paragraph further comprising automatically adjusting the hydrogen storage set point pressure, wherein the hydrogen storage set point pressure is a function of the flow rate of the hydrocarbon feed stream, the output of a primary pressure controller, or a combination of both. An embodiment of the present disclosure is one, any or all of prior embodiments in this paragraph up through the first embodiment in this paragraph further comprising comparing the pressure of the storage drum to the hydrogen storage set point pressure using a hydrogen storage pressure controller; passing the storage hydrogen stream to the storage drum based on a highest required flow from either of a secondary pressure controller and the hydrogen storage pressure controller; and discharging the stored hydrogen stream from the storage drum based on a highest required flow from either of a tertiary pressure controller and the hydrogen storage pressure controller.

A second embodiment of the present disclosure is a process of providing a hydrogen stream, comprising charging a hydrocarbon feed stream to a dehydrogenation reactor to provide a dehydrogenated stream; separating the dehydrogenated stream in a separator to provide a vapor stream comprising hydrogen and a liquid stream; compressing the vapor stream to provide a compressed vapor stream; taking a hydrogen product stream from the compressed vapor stream; comparing an outlet pressure of the hydrogen product stream in a hydrogen feed line with a set point pressure; adjusting a flow rate of the hydrocarbon feed stream to the dehydrogenation reactor based on a comparison of the outlet pressure with the set point pressure; and limiting the time rate of change in flow rate of hydrocarbon feed charged to the dehydrogenation reactor. An embodiment of the present disclosure is one, any or all of prior embodiments in this paragraph up through the second embodiment in this paragraph further comprising taking a storage hydrogen stream from the compressed vapor stream and controlling discharging a stored hydrogen stream from a storage drum in a storage discharge line when the outlet pressure is less than the set point pressure.

A third embodiment of the present disclosure is a process of providing a hydrogen stream, comprising charging a hydrocarbon feed stream to a dehydrogenation reactor to provide a dehydrogenated stream; separating the dehydrogenated stream in a separator to provide a vapor stream comprising hydrogen and a liquid stream; taking a hydrogen product stream from the vapor stream; comparing an outlet pressure of the hydrogen product stream in a hydrogen feed line with a set point pressure; adjusting a flow rate of the hydrocarbon feed stream to the dehydrogenation reactor based on a comparison of the outlet pressure with the set point pressure; taking a storage hydrogen stream from the vapor stream; passing the storage hydrogen stream to a storage drum in a storage inlet line when the outlet pressure exceeds the set point pressure; and discharging a stored hydrogen stream from the storage drum in a storage discharge line when the outlet pressure is less than the set point pressure. An embodiment of the present disclosure is one, any or all of prior embodiments in this paragraph up through the second embodiment in this paragraph further comprising adjusting a flow rate of said hydrocarbon feed stream to the dehydrogenation reactor based on a comparison of the outlet pressure with the set point pressure. An embodiment of the present disclosure is one, any or all of prior embodiments in this paragraph up through the second embodiment in this paragraph further comprising taking a storage hydrogen stream from said dehydrogenated stream. An embodiment of the present disclosure is one, any or all of prior embodiments in this paragraph up through the second embodiment in this paragraph further comprising discharging a stored hydrogen stream from said storage drum in a storage discharge line when the outlet pressure is less than said set point pressure.

A fourth embodiment of the present disclosure is a process of providing a hydrogen stream, comprising heating the hydrocarbon feed stream in a heater to provide a heated hydrocarbon feed stream; charging the heated hydrocarbon feed stream to the dehydrogenation reactor to provide a dehydrogenated stream comprising hydrogen; measuring an inlet temperature of the heated hydrocarbon feed stream to the reactor and an outlet temperature of the dehydrogenated stream from the reactor; calculating an average reactor temperature from a measured inlet temperature of the heated hydrocarbon feed stream and a measured outlet temperature of the dehydrogenated stream; and adjusting a duty of the heater when the average reactor temperature deviates from a set point average reactor temperature. An embodiment of the present disclosure is one, any or all of prior embodiments in this paragraph up through the first embodiment in this paragraph further comprising automatically adjusting an average reactor temperature set point, wherein the average reactor temperature set point is a function of a flow rate of the hydrocarbon feed stream. An embodiment of the present disclosure is one, any or all of prior embodiments in this paragraph up through the first embodiment in this paragraph further comprising a low selector switch in communication with the heater; and an override temperature controller in communication with the low selector switch, the override temperature controller overrides a temperature controller when a reactor inlet temperature exceeds a set point temperature. An embodiment of the present disclosure is one, any or all of prior embodiments in this paragraph up through the first embodiment in this paragraph further comprising limiting the time rate of change in a flow rate of the hydrocarbon feed charged to the dehydrogenation reactor.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of providing a hydrogen stream, comprising:
charging a hydrocarbon feed stream to a dehydrogenation reactor to provide a dehydrogenated stream;
taking a hydrogen product stream from said dehydrogenated stream;
comparing an outlet pressure of the hydrogen product stream in a hydrogen feed line with a set point pressure; and
adjusting a flow rate of said hydrocarbon feed stream to the dehydrogenation reactor based on a comparison of the outlet pressure with the set point pressure.

2. The process of claim 1 further comprising limiting the time rate of change in flow rate of said hydrocarbon feed stream charged to the dehydrogenation reactor.

3. The process of claim 1 further comprising:
taking a storage hydrogen stream from said vapor stream;
passing said storage hydrogen stream to a storage drum when the outlet pressure exceeds said set point pressure; and
discharging a stored hydrogen stream from said storage drum to said hydrogen product stream when the outlet pressure is less than said set point pressure.

4. The process of claim 1, wherein a primary pressure controller performs a measuring and the comparing steps for the outlet pressure of the hydrogen product stream.

5. The process of claim 3, wherein a secondary pressure controller measures the outlet pressure in the hydrogen feed line and admits said storage hydrogen stream to said storage drum when said outlet pressure exceeds said set point pressure, and wherein the secondary pressure controller has a higher set point than the primary pressure controller.

6. The process of claim 3, wherein a tertiary pressure controller measures the outlet pressure in the hydrogen feed line and discharges said stored hydrogen stream from said storage drum when said outlet pressure is inferior to said set point pressure.

7. The process of claim 6, wherein the tertiary pressure controller has a lower set point than the primary pressure controller.

8. The process of claim 5, wherein the secondary pressure controller 47 is in communication with a vent line to release a vent hydrogen stream when the outlet pressure of the storage drum exceeds said set point pressure.

9. The process of claim 8 further comprising
compressing said vapor stream in a recycle gas compressor to provide a compressed vapor stream;
taking said vent hydrogen stream in the vent line from said compressed vapor stream; and
taking said hydrogen product stream from said compressed vapor stream.

10. The process of claim 9, wherein said vent hydrogen stream is taken from said vapor stream before compressing said vapor stream.

11. The process of claim 2 further comprising:
heating said hydrocarbon feed stream in a heater to provide a heated hydrocarbon feed stream;
passing said heated hydrocarbon feed stream to the dehydrogenation reactor;
measuring an inlet temperature of said heated hydrocarbon feed stream to the reactor and an outlet temperature of said dehydrogenated stream from the reactor;
calculating an average reactor temperature from a measured inlet temperature of said heated hydrocarbon feed stream and a measured outlet temperature of said dehydrogenated stream; and
adjusting a duty of said heater when the average reactor temperature deviates from a set point average reactor temperature.

12. The process of claim 11 further comprising automatically adjusting an average reactor temperature set point, wherein the average reactor temperature set point is a function of said flow rate of said hydrocarbon feed stream.

13. The process of claim 11 further comprising:
a low selector switch in communication with the heater; and
an override temperature controller in communication with the low selector switch, the override temperature controller overrides a temperature controller when a reactor inlet temperature exceeds a set point temperature.

14. The process of claim 3 further comprising:
compressing at least a portion of said vapor stream in a compressor to provide said hydrogen product stream; and
discharging said stored hydrogen stream to an inlet of the compressor.

15. The process of claim 3 further comprising:
comparing a pressure of said storage drum to a hydrogen storage set point pressure;
passing said storage hydrogen stream to said storage drum when said storage drum pressure is below a hydrogen storage set point pressure; and
discharging said stored hydrogen stream from said storage drum when said storage drum pressure exceeds a hydrogen storage set point pressure.

16. The process of claim 15 further comprising:
automatically adjusting the hydrogen storage set point pressure, wherein the hydrogen storage set point pressure is a function of said flow rate of said hydrocarbon feed stream, the output of a primary pressure controller, or a combination of both.

17. The process of claim 15 further comprising:
comparing the pressure of said storage drum to the hydrogen storage set point pressure using a hydrogen storage pressure controller;
passing said storage hydrogen stream to said storage drum based on a highest required flow from either of a secondary pressure controller and said hydrogen storage pressure controller; and
discharging said stored hydrogen stream from said storage drum based on a highest required flow from either of a tertiary pressure controller and said hydrogen storage pressure controller.

18. A process of providing a hydrogen stream, comprising:
charging a hydrocarbon feed stream to a dehydrogenation reactor to provide a dehydrogenated stream;
taking a hydrogen product stream from said dehydrogenated stream;
comparing an outlet pressure of the hydrogen product stream in a hydrogen feed line with a set point pressure;
adjusting a flow rate of said hydrocarbon feed stream to the dehydrogenation reactor based on a comparison of the outlet pressure with the set point pressure; and
limiting the time rate of change in flow rate of hydrocarbon feed charged to the dehydrogenation reactor.

19. The process of claim 18 further comprising taking a storage hydrogen stream from said compressed vapor stream and controlling discharging a stored hydrogen stream from a storage drum in a storage discharge line when the outlet pressure is less than said set point pressure.

20. A process of providing a hydrogen stream, comprising:
charging a hydrocarbon feed stream to a dehydrogenation reactor to provide a dehydrogenated stream;
taking a hydrogen product stream from said dehydrogenated stream;
comparing an outlet pressure of the hydrogen product stream in a hydrogen feed line with a set point pressure; and
passing a storage hydrogen stream to a storage drum when the outlet pressure exceeds said set point pressure.
